(19) United States
(12) Patent Application Publication
(10) Pub. No.: US 2023/0020025 A1
(43) Pub. Date: Jan. 19, 2023

(54) DISPLAY ASSEMBLY FOR AN ELECTRONIC DEVICE THAT INCLUDES A BLUE LIGHT PROTECTIVE TRANSPARENCY

(71) Applicant: PPG Industries Ohio, Inc., Cleveland, OH (US)

(72) Inventors: Nicolas Benjamin Duarte, Allison Park, PA (US); Fatima Sophia Emami, Canoga Park, CA (US); John David McCall Shearer, Pittsburgh, PA (US); Maria Christina Baker, Los Angeles, CA (US)

(73) Assignee: PPG Industries Ohio, Inc., Cleveland, OH (US)

(21) Appl. No.: 17/757,578

(22) PCT Filed: Dec. 18, 2020
(86) PCT No.: PCT/US2020/065795

§ 371 Date: Jun. 17, 2022

Related U.S. Application Data
(60) Provisional application No. 62/950,622, filed on Dec. 19, 2019.

(51) Int. Cl. G02F 1/1335; H01J 31/20; G02B 5/20; H01L 27/32; C07D 487/04

(52) U.S. Cl. CPC G02F 1/133514; H01J 31/20; G02B 5/208; H01L 27/322; C07D 487/04; G02F 1/133528; G02F 1/13338

(57) ABSTRACT

A display screen assembly is provided comprising a blue light protection transparency. The transparency comprises an additive, such that the blue light protection transparency blocks greater than 50% of light of a wavelength range of 380 nm to 418 nm from passing through the blue light protection transparency as measured by ultraviolet-visible spectroscopy; and the blue light protection transparency blocks 20% to 45% of light of a wavelength range of greater than 418 nm to 430 nm from passing through the blue light protection transparency as measured by ultraviolet-visible spectroscopy. Additionally, the blue light protection transparency has a total visible light transmittance greater than 65% as measured according to ASTM D1003-07 Procedure B.

### FIELD OF THE INVENTON

The present disclosure relates to display screen assemblies comprising a blue light protection transparency. The assemblies are particularly useful for electronic and other devices.

### BACKGROUND OF THE INVENTION

Recent studies on sleep by researchers at Harvard University have shown that even small amounts of high energy visible (HEV, or “blue”) light emitted from screens can have a powerful and detrimental impact on sleep by inhibiting melatonin production in the body and altering circadian rhythms. Blue light is emitted from many common household electronic devices such as TVs, computers, laptops, smart phones, tablets, and fluorescent and LED lighting.

Compared to other colors on the visible spectrum, blue light has a short wave-length and hence is high energy, acting as a potent suppressor of sleep-inducing melatonin.

Blue light exposure during the evening, particularly immediately prior to bedtime, increases the time it takes to fall asleep due to heightened alertness. Blue light exposure also impairs sleep quality by reducing REM sleep (i. e., dream sleep) and deep phase sleep, which in turn make it more difficult to wake up.

It would be desirable to provide display screen assemblies for electronic and other devices that minimize exposure to blue light.

### SUMMARY OF THE INVENTION

An electronic display screen assembly is provided comprising a blue light protection transparency. The transparency comprises an additive, such that the blue light protection transparency blocks greater than 50% of light of a wavelength range of 380 nm to 418 nm from passing through the blue light protection transparency as measured by ultraviolet-visible spectroscopy; and the blue light protection transparency blocks 20% to 45% of light of a wavelength range of greater than 418 nm to 430 nm from passing through the blue light protection transparency as measured by ultraviolet-visible spectroscopy. Additionally, the blue light protection transparency has a total visible light transmittance greater than 65% as measured according to ASTM D1003-07 Procedure B.

### BRIEF DESCRIPTION OF THE DRAWINGS

FIGS. 1A to 1F are schematic representations of various exemplary layers in an LCD display screen assembly in accordance with the present invention, demonstrating that the blue light protection transparency may be situated in any of various locations in the assembly stack.

FIGS. 2A to 2C are schematic representations of various exemplary layers in an OLED display screen assembly in accordance with the present invention, demonstrating as an example that the blue light protection transparency may be used as a replacement for the glass substrate upon which the OLED is built or as a protective component for the display between the emission source and the user/viewer.

FIGS. 3A and 3B are schematic representations of various exemplary layers in a CRT display screen assembly in accordance with the present invention, demonstrating as an example that the blue light protection transparency may be used as a replacement for the glass substrate upon which a phosphorus emission layer is formed or as a protective component for the display between the emission source and the user/viewer.

FIGS. 4A to 4B are schematic representations of various exemplary layers in a touch screen display assembly in accordance with the present invention. The blue light protection transparency may be used, for example, as a replacement for either one of the hard coat layers shown.

### DETAILED DESCRIPTION

The present disclosure is directed toward an electronic display screen assembly comprising a transparency that blocks at least a portion of light of a wavelength range of 380 nm to 430 nm. The transparency comprises an additive, such that the blue light protection transparency blocks greater than 50% of light of a wavelength range of 380 nm to 418 nm from passing through the blue light protection transparency as measured by ultraviolet-visible spectroscopy; and the blue light protection transparency blocks 20% to 45% of light of a wavelength range of greater than 418 nm to 430 nm from passing through the blue light protection transparency as measured by ultraviolet-visible spectroscopy. Additionally, transparencies as described herein have a total visible light (i. e., light having a wavelength of from about 380 to 750 nm) transmittance of greater than 65% (such as greater than 70%, greater than 75%, greater than 80%, greater than 85%, greater than 90%, or greater than 95%) as measured according to ASTM D1003-07 Procedure B. This high visible light transmittance provides aesthetic advantages, such as a more natural-looking display. Studies have also demonstrated that blocking light in the wavelength range of 380 nm to 430 nm from being transmitted through a transparency reduces the risk of melatonin depletion, cancer, and/or ocular damage (e.g., macular degeneration, cataracts, etc.) for organisms in the vicinity of the transparency, by decreasing the total exposure of the organism to damaging light (e.g., light having a wavelength of 380 nm to 430 nm).

The blue light protection transparency is understood to be transparent, by which is meant that the transparency exhibits a haze value of less than 5 percent, e.g., less than 1 percent or less than 0.5 percent, when the haze value is measured at 550 nanometers by, for example, by Color i7 spectrophotometer from X-Rite, Inc.

As described herein, the amount of light of a wavelength range that is blocked refers to an average amount of light of the entire wavelength range that is blocked. This may include that the amount of light at each wavelength of the wavelength range that is blocked is as high as well. For example, a transparency that blocks greater than 50% of light of a wavelength range of 380 nm to 418 nm, may block an average of greater than 50% of light of the wavelength range of 380 nm to 418 nm (e.g., where the % blockage is calculated from the average across the wavelength range of 380 nm to 418 nm), and may optionally block greater than 50% of the light at each wavelength of the range of 380 nm to 418 nm as well.

As described herein, the amount of light “blocked” by the transparency refers to, for the given wavelength range, the amount of light that is incident to the transparency and not transmitted (e.g., not fully transmitted) through the transparency. The remaining portion of the light may be transmitted (e.g., fully transmitted) through the transparency. For example, for a transparency that blocks greater than 50% of light of the wavelength range of 380 nm to 418 nm, at most 50%, for the given wavelength range and optionally also for each wavelength within the range, of the light that is incident to the transparency is transmitted through the transparency (e.g., transmitted from a first side of the transparency to a second side of the transparency facing the first side). The greater than 50% of the light of the wavelength range of 380 nm to 418 nm may be blocked by any suitable mechanism such as, for example, light absorption, light reflection, light refraction, or any other suitable mechanism, but the present disclosure is not limited thereto.

As used herein, the terms “wavelength range of” and “in the range of” include the end points of the range (e.g., 380 nm and 430 nm) in addition to the values between the end points. Additionally, the transparency described herein may block up to 60%, 65%, 70%, 75%, 80%, 85%, 90%, 95%, 96%, 99%, 99.5%, 99.9%, 99.99%, 99.999%, or 100% of the light of the wavelength range described. The amount of light blocked by the transparency was measured utilizing ultraviolet-visible spectroscopy, which may also be referred to as ultraviolet-visible spectrophotometry, UV-Vis, or UV/Vis. For example, the amount of light blocked may be determined from the average of the amount of light blocked of the given wavelength range, from the amount of light blocked at each wavelength of the given wavelength range, or from both, as measured by ultraviolet-visible spectroscopy. Thus, the transparency blocks greater than 50% of light of the wavelength range of 380 nm to 418 nm and 20% to 45% of light of a wavelength range of greater than 418 nm to 430 nm, as measured by ultraviolet-visible spectroscopy. Additionally, the total visible light transmittance described herein (e.g., a total visible light transmittance of greater than 65%), was measured by way of ASTM D1003-07 Procedure B.

The transparency may also block at least a portion of other light in addition to the light of the wavelength range of 380 nm to 430 nm. For example, the transparency may block greater than 70% (or greater than 75%, 80%, 85%, 90%, 95%, 99.0%, 99.5%, or 99.9%) of ultraviolet C light (UVC, e.g., light of a wavelength range of 100 nm to 280 nm), may block greater than 70% (or greater than 75%, 80%, 85%, 90%, 95%, 99.0%, 99.5%, or 99.9%) of ultraviolet B light (UVB, e.g., light of a wavelength range of 280 nm to 315 nm), and/or may block greater than 70% (or greater than 75%, 80%, 85%, 90%, 95%, 99.0%, 99.5%, or 99.9%) ultraviolet A light (UVA, e.g., light of a wavelength range of 315 nm to 400 nm). The transparency may block greater than 70% (or 75%, 80%, 85%, 90%, 95%, 99%, 99.5%, or 99.9%) of light of a wavelength range of 100 nm to 315 nm from passing through the transparency.

Because the amount of light blocked may be an average of the light blocked across a wavelength range, the amount of light blocked across a subrange subsumed within a broader range may be different from the amount of light blocked across the broader range. For example, a transparency that blocks greater than 50% of light of the wavelength range of 380 nm to 418 nm may block greater than 45% (or greater than 50%, greater than 55%, greater than 75%, greater than 80%, or greater than 95%) of light of a wavelength range of 400 nm to 410 nm. The transparency may block greater than 40% (or greater than 55%, greater than 90%, greater than 95%, or greater than 98%) of light of a wavelength range of 380 nm to 410 nm. The transparency may block greater than 60% (or greater than 64%, greater than 70%, greater than 90%, greater than 95%, greater than 96%, or greater than 99%) of light of a wavelength range of 200 nm to 418 nm. The transparency may additionally block any suitable amount of light having a wavelength greater than 430 nm (e.g., violet light of a wavelength range of 430 nm to 450 nm, and/or blue light of a wavelength range of 450 nm to 490 nm).

In the present disclosure, the numerical ranges described include all subranges subsumed therein. For example, the wavelength range of 400 nm to 430 nm includes a wavelength range of 400 nm to 420 nm, or any wavelength range including 400 nm, 401 nm, 402 nm, 403 nm, 404 nm, 405 nm, 406 nm, 407 nm, 408 nm, 409 nm, 410 nm, 411 nm, 412 nm, 413 nm, 414 nm, 415 nm, 416 nm, 417 nm, 418 nm, 419 nm, 420 nm, 421 nm, 422 nm, 423 nm, 424 nm, 425 nm, 426 nm, 427 nm, 428 nm, 429 nm, or 430 nm as a starting point or end point of the wavelength range.

Additionally, each of the light blockage ranges described herein includes all subranges subsumed therein. For example, a transparency that blocks greater than 50% of the light of the wavelength range of 380 nm to 418 nm may block any amount of light in the range of 50% to 100%, as measured by ultraviolet-visible spectroscopy. The transparency may block greater than 60%, greater than 61%, greater than 65%, greater than 70%, greater than 71%, greater than 72%, greater than 73%, greater than 74%, greater than 75%, greater than 80%, greater than 85%, greater than 90%, greater than 95%, greater than 99%, greater than 99.1%, greater than 99.2%, greater than 99.3%, greater than 99.4%, greater than 99.5%, greater than 99.6%, greater than 99.7%, greater than 99.8%, greater than 99.9%, greater than 99.98%, or greater than 99.99% of the light of the wavelength range of 380 nm to 418 nm, as measured by ultraviolet-visible spectroscopy, where the upper limit of each of the foregoing is 100%.

As noted above, by blocking light of the wavelength range of 380 nm to 430 nm from being transmitted through the transparency, the transparency reduces the risk of melatonin depletion, cancer, and/or ocular damage (e.g., macular degeneration, cataracts, etc.) for organisms in the vicinity of the transparency, by decreasing the total exposure of the organism to damaging light (e.g., light having a wavelength of 380 nm to 430 nm). For example, the transparency reduces the exposure of a user of an electronic device with a display screen assembly including the transparency to light of a wavelength range of 400 nm to 430 nm.

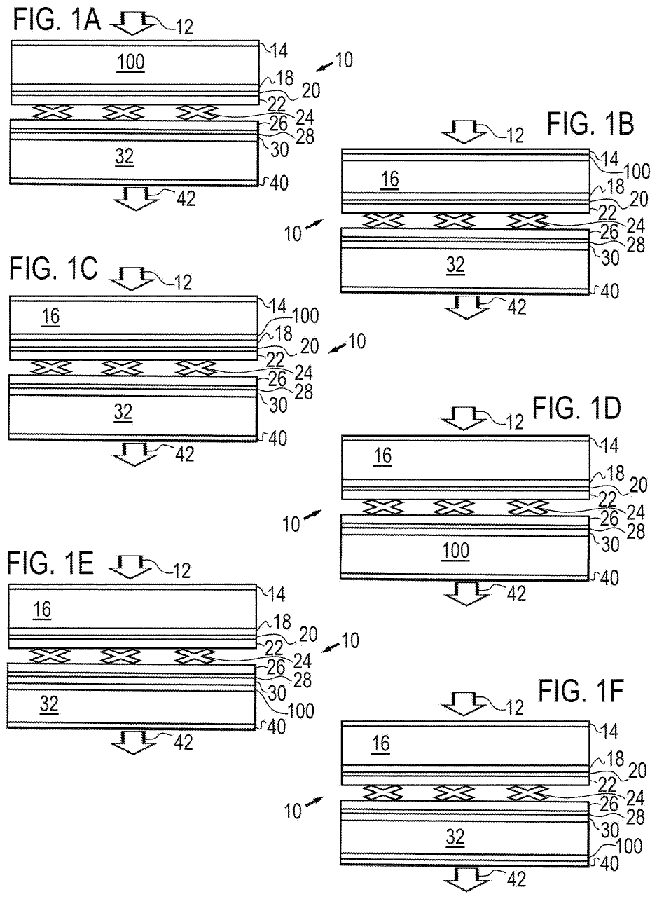

The blue light protection transparencies described above may be used as a component in a display screen assembly in accordance with the present invention. The display screen assembly may comprise, for example, an active or passive liquid crystal cell element; a light emitting diode (LED, including OLED); or a cathode ray tube (CRT). The display screen assembly may serve as a display screen or a touch screen on an electronic device such as a cell phone, tablet, GPS, voting machine, interactive toy, digital camera, diagnostic tool, medical device, smart watch, gauge cluster, digital instrumentation display, or POS (Point-Of-Sale) machine; a computer (e. g., laptop) screen; interactive advertising display screen, transparent see-through OLED display; electronic video display wall; smart appliance display screen, vehicle infotainment display screen, or a monitor screen. For example, as shown in FIGS. 1A to 1F, a blue light protection transparency 100 may be used in a liquid crystal display (LCD) assembly 10 in accordance with the present invention, in addition to or in place of one or more other layers in the assembly, such as 1) in the light path between light source 12 and liquid crystal component 24 such that the blue light protection transparency 100 acts as the substrate, pre-substrate, or post substrate; or 2) in the light path between the liquid crystal component 12 and the user including the substrate or post substrate area (including a protective component for the display). In FIG. 1A, the blue light protection transparency 100 replaces a glass substrate as follows: unpolarized white light from light source 12 passes through a first polarizer 14, the blue light protection transparency 100 (which takes the place of a glass substrate that is situated in this position in a conventional LCD display assembly), then through a thin film transistor (TFT) 18, a first indium tin oxide (ITO) film 20, a first orientation film 22, liquid crystals 24, a second orientation film 26, a second ITO film 28, color filter 30, glass substrate 32, a second polarizer 40, and then exits the LCD display assembly 10 as polarized light 42. In FIG. 1B, the blue light protection transparency 100 is positioned in an LCD display assembly 10 just prior to a glass substrate 16. In FIG. 1C, the blue light protection transparency 100 is positioned in an LCD assembly 10 just after a glass substrate 16. In FIG. 1D, the blue light protection transparency 100 replaces a glass substrate as follows: unpolarized white light from light source 12 passes through a first polarizer 14, a glass substrate 16, then through TFT 18, a first ITO film 20, a first orientation film 22, liquid crystals 24, a second orientation film 26, a second ITO film 28, color filter 30, the blue light protection transparency 100 (which takes the place of a glass substrate that is situated in this position in a conventional LCD display assembly), a second polarizer 40, and then exits the LCD display 10 as polarized light 42. In FIG. 1E, the blue light protection transparency 100 is positioned in an LCD assembly 10 just prior to a glass substrate 32. In FIG. 1F, the blue light protection transparency 100 is positioned in an LCD assembly 10 just after a glass substrate 32.

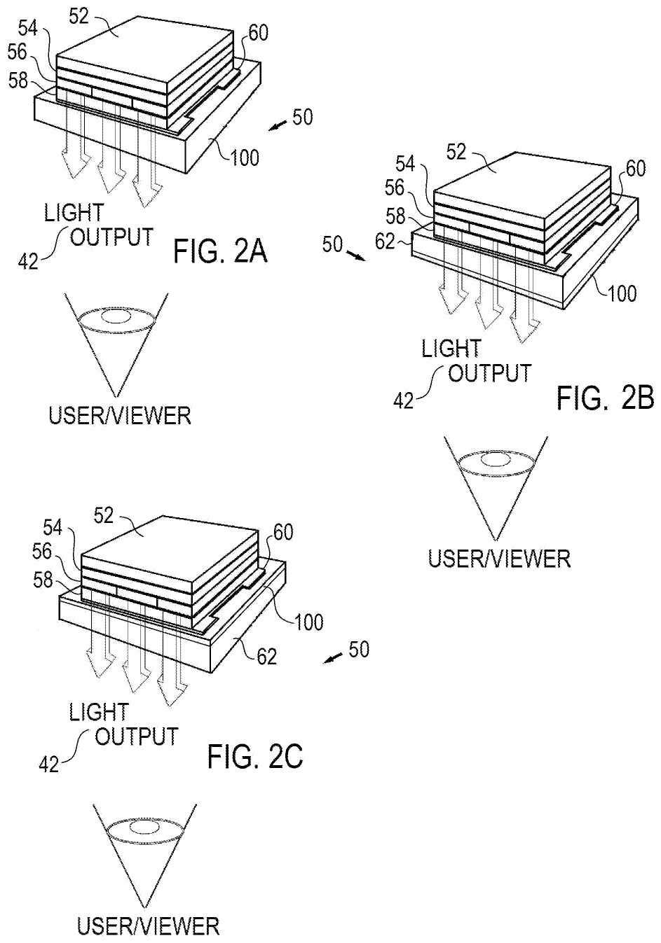

As shown in FIGS. 2A to 2C, the blue light protection transparency 100 may be used in an OLED display assembly 50 in accordance with the present invention, for example as a replacement for a glass substrate upon which the OLED display assembly 50 is built or as a protective component for the OLED display assembly 50 between an emission source and the user/viewer. In FIG. 2A, the blue light protection transparency 100 replaces a glass substrate as follows: The OLED display assembly 50 comprises a metal cathode 52, electron transport layer 54, organic emitters 56, hole injection layer 58, anode 60, and the blue light protection transparency 100 (which takes the place of a glass substrate that is situated in this position in a conventional OLED display assembly). Light exits the OLED display assembly 50 as light output 42, toward a viewer. In FIG. 2B, the blue light protection transparency 100 is positioned in an OLED assembly 50 just after a glass substrate 62. In FIG. 2C, the blue light protection transparency 100 is positioned in an OLED display assembly 50 just prior to a glass substrate 62.

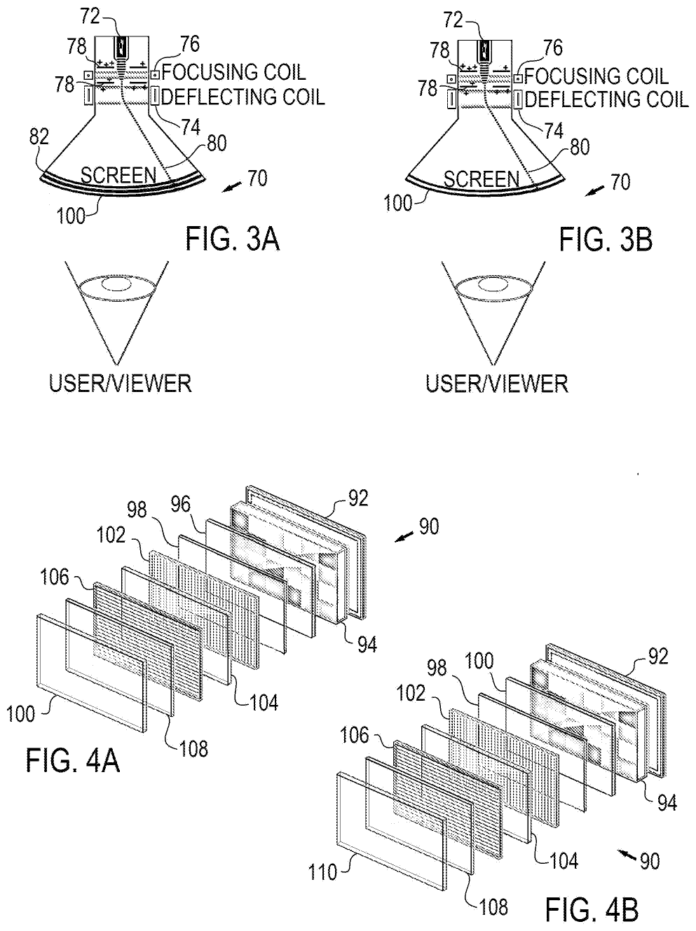

As shown in FIGS. 3A and 3B, the blue light protection transparency 100 may be used in a CRT display assembly 70 in accordance with the present invention, for example as a replacement for a glass substrate (screen) upon which a phosphorus emission layer may be formed or as a protective component for the CRT display assembly 70 between an emission source and the user/viewer. In FIG. 3A, the blue light protection transparency 100 is positioned after a screen as follows: The CRT display assembly 70 comprises a cathode 72 from which an electron beam 80 is emitted, a focusing coil 76, a deflecting coil 74, anodes 78, screen 82, and the blue light protection transparency 100. In FIG. 3B, the blue light protection transparency 100 takes the place of a screen that is situated in this position in a conventional CRT display assembly.

As shown in FIGS. 4A and 4B, the blue light protection transparency 100 may be used in a touch screen display assembly 90 in accordance with the present invention, for example acting as a “hard coat” layer between a sensing layer and an LCD or as a hard coat layer between the sensing layer and the user. In FIG. 4A, the blue light protection transparency 100 replaces a hard coat as follows: The touch screen display assembly 90 comprises a case 92, an LCD 94, a hard coat 96, a first adhesive layer 98, a sensing layer 102, an ITO layer 104, an emitting layer 106, a second adhesive layer 108, and the blue light protection transparency 100 (which takes the place of a hard coat that is situated in this position in a conventional touch screen display assembly). In FIG. 4B, the blue light protection transparency 100 replaces a hard coat as follows: The touch screen display assembly 90 comprises a case 92, an LCD 94, the blue light protection transparency 100 (which takes the place of a hard coat that is situated in this position in a conventional touch screen display assembly), a first adhesive layer 98, a sensing layer 102, an ITO layer 104, an emitting layer 106, a second adhesive layer 108, and a hard coat 110.

As is known in the art, depending on the nature of the display screen assembly, the assembly may comprise multiple layers that serve different purposes in the assembly layer stack. The blue light protection transparency may be situated during manufacturing of the display screen assembly as an outermost layer or an inner or innermost layer. It may be included as an additional component of a conventional display screen assembly for ease of manufacture, situated anywhere within the assembly layer stack provided it does not interfere with the function of the other layers. In an LCD assembly, for example, the blue light protection transparency may be situated in the light path between the light source and liquid crystal component, or in the light path between the liquid crystal component and the user. Alternatively, the blue light protection transparency may replace conventional display screen layers, such as glass substrates typically used in LCD, OLED, and CRT DLP, reflective system assemblies, or hard coat layers used in touch screen assemblies. The blue light protection transparency may also be used in conjunction with a conventional hard coat layer, which can be applied to any layer in the assembly.

In a simplified example, the display screen assembly may comprise at least a first substrate and a second substrate, with an interlayer between the first substrate and the second substrate, where the interlayer comprises the blue light protection transparency. For example, a touch screen assembly may comprise an LCD as a first substrate and a sensing layer as a second substrate, with an interlayer between them comprising the blue light protection transparency.

The blue light protection transparency may serve multiple purposes within the display screen assembly; for example, the additive may be incorporated into or coated on a polarizing layer in an LCD assembly, such that the polarizing layer is also a blue light protection transparency. Often, the transparency demonstrates a #0000 steel wool abrasion resistance (1 kg load, water contact angle >100°) of at least 2000 cycles, or at least 2500 cycles, or at least 3000 cycles, or at least 6000 cycles, or at least 12000 cycles, or at least 20000 cycles. This is particularly desirable when the blue light protection transparency is an outermost layer of the display screen assembly, to help prevent scratching of the display screen.

The blue light protection transparency may block light of the wavelength range of 380 nm to 430 nm by including the additive, which may include light blocking compounds and/or light absorbing compounds (e.g., compounds that block, reflect, refract, and/or absorb light having a wavelength of 380 nm to 430 nm). The additive may include additional compounds such as, for example, light stabilizers that scavenge radicals resulting from the absorption of light (e.g., light having a wavelength of 400 nm to 430 nm) by other components of the transparency. The additive may be included in the transparency in any amount suitable for achieving the amount of light blocking described herein. For example, the additive (e.g., the light blocking compounds and/or light absorbing compounds) may be included in an amount of 0.00001 wt % to 10 wt % (or any range subsumed therein), based on the total weight of the transparency. The additive (e.g., the light blocking compounds and/or light absorbing compounds) may be included in a composition for forming the transparency in an amount of 0.00001 wt % to 10 wt % (or any range subsumed therein), based on the total weight of the solids of a composition for forming a layer of the transparency. For example, the additive (e.g., the light blocking compounds and/or light absorbing compounds) may be included in either of the foregoing in an amount of 0.00001 wt % to 0.01 wt %; 0.001 wt % to 5 wt %; 0.01 wt % to 2 wt %; 0.01 wt % to 1.9 wt %; 0.1 to 1.8 wt %; 0.3 wt % to 1.5 wt %; 0.5 wt % to 1.3 wt %; 0.6 wt % to 1.25 wt %; 0.9 wt % to 1.2 wt %; 0.01 wt % to 0.02 wt %; 0.02 wt % to 0.04 wt %; 2 wt % to 5 wt %; or 3 wt % to 4 wt %), based on the total weight of the transparency or the total weight of solids in the composition for forming a layer of the transparency.

Examples of the additive include classes of chemical compounds including: inorganic nanoparticles (e.g., metal oxides and metal nanoparticles), organic nanoparticles, organometallic nanoparticles, benzotriazoles, triazines, benzophenones, hindered amine light stabilizers (HALS), benzoates, cyanoacrylates, tetraphenylporphyrins, tetramesitylporphyrins, perylenes, oxalanilides, phthalocyanines, chlorophylls (including derivatives thereof), bilirubin (including derivatives thereof), primary antioxidants, pigments dyes (e.g., organometallic dyes), and combinations thereof. Non-limiting examples of the dye include bilirubin; chlorophyll a, diethyl ether; chlorophyll a, methanol; chlorophyll b; diprotonated-tetraphenylporphyrin; hematin; magnesium octaethylporphyrin; magnesium octaethylporphyrin (MgOEP); magnesium phthalocyanine (MgPc), PrOH; magnesium phthalocyanine (MgPc), pyridine; magnesium tetramesitylporphyrin (MgTMP); magnesium tetraphenylporphyrin (MgTPP); octaethylporphyrin; phthalocyanine (Pc); porphin; tetra-t-butylazaporphine; tetra-t-butylnaphthalocyanine; tetrakis(2,6-dichlorphenyl)porphyrin; tetrakis(o-aminophenyl)porphyrin; tetramesitylporphyrin (TMP); tetraphenylporphyrin (TPP); vitamin B12; zinc octaethylporphyrin (ZnOEP); zinc phthalocyanine (ZnPc), pyridine; zinc tetramesitylporphyrin (ZnTMP); zinc tetramesitylporphyrin radical cation; zinc tetraphenylporphyrin (ZnTPP); perylene; oxanilide; derivatives thereof; and combinations thereof.

The additive may be chosen from metal oxides, benzotriazoles (including derivatives thereof), triazines (including derivatives thereof), triazoles (including derivatives thereof), hindered amine light stabilizers (HALS, including derivatives thereof), silanes having amine functionality (including derivatives thereof), sterically hindered phenolic antioxidants (including derivatives thereof), silanes having isocyanate functionality (including derivatives thereof), and mixtures thereof. The inorganic nanoparticles may include a metal oxide chosen from cerium oxide (e.g., CeO₂), zinc oxide (e.g., ZnO), and the like, but the inorganic nanoparticles are not limited thereto. The additive may include a benzotriazole a triazine, a dye, a hindered amine light stabilizer, or a combination thereof in an amount of 0.01 wt % to 2.0 wt %, based on the total weight of the transparency. For example, the additive may include the benzotriazole, the dye, the hindered amine light stabilizer, or the combination thereof in an amount of 0.01 wt % to 1.5 wt %, based on the total weight of the transparency.

The additive may include a pyrrolo[3,4-f]benzotriazole-5,7(2H,6H)-dione. For example, a non-limiting example of the additive includes 6-butyl-2-[2-hydroxy-3-(1-methyl-1-phenylethyl)-5-(1,1,3,3-tetramethylbutyl)phenyl] pyrrolo[3,4-f]benzotriazole-5,7(2H,6H)-dione (commercially available as TINUVIN® CARBOPROTECT® (each available from BASF SE of Ludwigshafen, Germany). Additional, non-limiting commercial examples of the benzotriazole include TINUVIN® 99-2, TINUVIN® 384-2, TINUVIN® 900, and TINUVIN® 1130 (each available from BASF SE of Ludwigshafen, Germany), and CHIGUARD R-455 available from Chitec Technology Co., Ltd. of Taipei, Taiwan. Non-limiting commercial examples of the HALS include TINUVIN® 123, TINUVIN® 144, and TINUVIN® 292, each available from BASF SE of Ludwigshafen, Germany. Non-limiting commercial examples of the triazine include TINUVIN® 400, TINUVIN® 405, TINUVIN® 460, TINUVIN® 477, and TINUVIN® 479, each available from BASF SE of Ludwigshafen, Germany. Non-limiting commercial examples of the dye (e.g., the organometallic dye) include Cu(II) Meso—tetra (4-carboxyphenyl) porphine (e.g., High Performance Optics Dye Generation4D, available from High Performance Optics of Roanoke, Va., or any other suitable High Performance Optics Dye, including Generation 4A, 4B, and/or 4C).

Non-limiting commercial examples of the additive may also include: a TINUVIN® compound (e.g., TINUVIN® 151, TINUVIN® 152, TINUVIN® 213, TINUVIN® 234, TINUVIN® 326, TINUVIN® 327, TINUVIN® 328, TINUVIN® 571, TINUVIN® 622, TINUVIN® 765, TINUVIN® 770, and/or TINUVIN® P; each available from BASF SE of Ludwigshafen, Germany), an IRGANOX® compound (e.g., IRGANOX® 245, IRGANOX® 1010, IRGANOX® 1035, IRGANOX® 1076, IRGANOX® 1098, IRGANOX® 1135, and/or IRGANOX® 5057; each available from BASF SE of Ludwigshafen, Germany), Unitex OB (available from Angene Chemica of Hong Kong), a CHIMASSORB® compound (e.g., CHIMASSORB® 81, CHIMASSORB® 944 LD, and/or CHIMASSORB® 2020 FLD; each available from BASF SE of Ludwigshafen, Germany), a BLS® compound (e.g., BLS® 99-2, BLS® 119, BLS® 123, BLS® 234, BLS® 292, BLS® 531, BLS® 0113-3, BLS® 1130, BLS® 1326, BLS® 1328, BLS® 1710, BLS® 2908, BLS® 3035, BLS® 3039, and/or BLS® 5411; each available from Mayzo Inc. of Suwanee, Ga., USA), and/or CYASORB CYNERGY SOLUTIONS® L143-50X Stabilizer (available from Cytec Industries, Inc. of Woodland Park, N.J., USA).

The additive may include TINUVIN® CARBOPROTECT®, TINUVIN® 477 (which includes a red-shifted Tris-Resorcinol-Triazine Chromophore), compounds available from High Performance Optics of Roanoke, Va. (e.g., Generation 4B dye and/or Generation 4D dye), TINUVIN® 292, and/or TINUVIN® 1130. The additive may be included in the transparency, for example, by mixing with the polymer precursors for forming the transparency, at a wt % of 0.00001 wt % to 10 wt % (e.g., 0.001 wt % to 5 wt %; 0.01 wt % to 2 wt %; 0.01 wt % to 1.9 wt %; 0.1 to 1.8 wt %; 0.3 wt % to 1.5 wt %; 0.5 wt % to 1.3 wt %; 0.6 wt % to 1.25 wt %; 0.9 wt % to 1.2 wt %; 0.01 wt % to 0.02 wt %; 0.02 wt % to 0.04 wt %; 2 wt % to 5 wt %; or 3 wt % to 4 wt %), based on the total weight of the transparency or the total weight of the solids in a composition for forming a layer of the transparency.

The additive may include a functional group that facilitates incorporation of the additive into the transparency, for example, by improving chemical integration of the additive into the chemical backbone of a component or layer of the transparency during polymerization. The functional group may be chosen from amines, alcohols, or other functional groups that impart reactive functionality to the additive and do not inhibit the light absorptive or protective properties of the additive. The functional group may also tether the additive to the chemical backbone of the transparency to prevent or reduce migration of the additive to a surface of the transparency, thereby preventing or reducing blooming as the product ages. Additionally, by reducing or preventing migration of the additive to a surface of a layer of the transparency, the properties of the layer (e.g., adhesiveness, smoothness, etc.) may be relatively unaffected by the presence of the additive in the layer. As such, the layer including the additive may have substantially the same surface properties as a layer having substantially the same composition without the additive. This may be particularly desirable in a touch screen assembly. In a non-limiting example, the additive includes Cu(II) Meso—tetra (4-carboxyphenyl) porphine (commercially available as Generation 4D dye), which has four carboxylic groups. While the present disclosure is not limited by any particular mechanism or theory it is believed that the carboxylic groups reduce the likelihood or amount of migration of the additive and/or blooming.

The additive may be incorporated into the transparency in any suitable manner. For example, the additive may be included in a resin for forming a layer of or coating on the transparency. The additive may be added to a component for forming the resin and/or may be added to the resin directly. Further, the additive may be added to the resin before and/or during polymerization (e.g., curing). The resin may be polymerized to directly form the layer. The resin may be polymerized to form a polymerized product, which may be granulated, melt-processed, and extruded to form a layer. The additive may be added before and/or during the resin curing, and/or during the melt processing. For example, the additive may be dispersed into the polyol portion of a polyurethane interlayer formulation prior to polymerization, and/or the additive may be added to the polymer granulate during the extrusion process.

The blue light protection transparency may comprise any suitable material such as, for example, transparent ceramic, glass and/or polymer (e.g., plastic). The glass of the transparency may include any suitable glass such as, for example, clear float glass, water white glass, soda lime glass, lithium aluminosilicate glass, window glass, unibatch glass, borosilicate glass, heat absorbing blue and/or green glass, and low iron glass (e.g., STARPHIRE® glass commercially available from Vitro Architectural Glass of Cheswick, Pa.). Non-limiting commercial examples of the glass may include CORNING® GORILLA® glass (available from Corning Inc. of Corning, N.Y.), SOLIDION® glass (available from Saint-Gobain Sully of Courbevoie, France), Chemplex 2000 (available from GKN Aerospace of Phoenix, Ariz.). The glass may be tempered (e.g., chemically tempered and/or thermally tempered). Non-limiting commercial examples of chemically tempered glass include such HERCULITE® or HERCULITE® II glass available from PPG of Pittsburgh, Pa. A polymer of the transparency may include polycarbonate, acrylic, stretched acrylic, allyl diglycol carbonate (e.g., CR-39, available from PPG of Pittsburgh, Pa.), polyurethane (e.g., S-123 available from PPG of Pittsburgh, Pa.), plastic (e.g., OPTICOR® ATM, available from PPG of Pittsburgh, Pa.), polyurea, polyvinyl, polystyrene, cyclic olefin copolymers, poly-4-methylpentane, amorphous nylon and other polyamides, poly(m)ethylmethacrylates, silicone, and/or polysulfones such as those sold under the names RADEL and UDEL by Solvay. Often the blue light protection transparency comprises a polyurethane sheet.

The transparency may have any suitable thickness. The thickness of the transparency may affect the light transmittance and % light blockage of the transparency, and therefore, the thickness of the transparency may be tailored to the desired properties. For example, the transparency may have a thickness of 0.5 mm to 25 mm (e.g 0.5 mm to 5 mm, 1 mm to 4 mm, or 2 mm to 3 mm).

The concentration of the additive in the blue light protection transparency may be varied depending upon the thickness of the transparency cross-section, and/or the composition of the transparency. Relatively lower concentrations (e.g., 0.00001 wt % to 5 wt %) of additives (e.g., the light blocking compounds and/or light absorbing compounds) may be utilized for transparencies including S-123 and/or OPTICOR® ATM, each available from PPG of Pittsburgh, Pa.

The additive may be incorporated directly into a substrate used to prepare the transparency. The additive may be added to a monomer mixture used to prepare a polymeric substrate, or post-added to a polymer that will be formed into the substrate. The substrate comprising a polyurethane may include a reaction product of components including an isocyanate and an aliphatic polyol having 4 to 18 carbon atoms (or any range subsumed therein, for example, 5 to 17 carbon atoms, 6 to 16 carbon atoms, 7 to 15 carbon atoms, 8 to 14 carbon atoms, or 9 to 13 carbon atoms) and 2 hydroxyl groups (or 3 or more hydroxyl groups), wherein the isocyanate and/or the aliphatic polyol may be branched or unbranched. As used herein, the term “an isocyanate” may mean “at least one polyisocyanate” and the term “an aliphatic polyol” may mean “at least one aliphatic polyol.” According to the present disclosure, the isocyanate and/or the polyol may be branched. As used herein, the term “branched” means a chain of atoms with one or more side chains attached to it. Branching occurs by the replacement of a substituent, e.g., a hydrogen atom, with a covalently bonded functional substituent or moiety, e.g., an hydroxyalkyl group. As used herein, the term “isocyanate” means a compound including an —N═C═O functional group and/or an —N═C═S (isothiocyanate) group and is construed broadly to include polyisocyanates that include two or more —N═C═O functional groups and/or two or more —N═C═S (isothiocyanate) groups, such as diisocyanates, triisocyanates, or higher functional isocyanates, as well as dimers and trimers or biurets of isocyanates. The isocyanate may be capable of forming a covalent bond with a reactive group such as a hydroxyl, thiol, or amine functional group. Branched isocyanates may be used to increase the free volume within a polymer matrix formed by the polyurethane to provide space for the molecules to move.

Non-limiting examples of suitable isocyanates include aliphatic, cycloaliphatic, aromatic, heterocyclic, sulfonate containing, and/or self-healing molecules, e.g., alkoxyamines, dimers and trimers thereof, and mixtures thereof. Non-limiting examples of suitable cycloaliphatic isocyanates include those in which one or more of the isocyanate groups are attached directly to the cycloaliphatic ring and cycloaliphatic isocyanates in which one or more of the isocyanate groups are not attached directly to the cycloaliphatic ring. Non-limiting examples of suitable aromatic isocyanates include those in which an isocyanate group is attached directly to the aromatic ring, and aromatic isocyanates in which an isocyanate group is not attached directly to the aromatic ring. Non-limiting examples of suitable heterocyclic isocyanates include those in which an isocyanate group is attached directly to the heterocyclic ring and heterocyclic isocyanates in which an isocyanate group is not attached directly to the heterocyclic ring. Non-limiting examples of suitable isocyanates may include DESMODUR W (4,4′-methylene-bis-(cyclohexyl isocyanate) or 1-Isocyanato-4-[(4-isocyanatocyclohexyl)methyl]cyclohexane), DESMODUR N 3300 (hexamethylene diisocyanate trimer), and DESMODUR N 3400 (60% hexamethylene diisocyanate dimer and 40% hexamethylene diisocyanate trimer), which are commercially available from Bayer Corp. of Pittsburgh, Pa.

As used herein, the term “polyol” includes compounds, monomers, oligomers, and polymers including at least two hydroxyl groups, e.g., including two hydroxyl groups (such as diols) or three hydroxyl groups (such as triols), higher functional polyols, and mixtures thereof. Non-limiting suitable polyols are capable of forming a covalent bond with a reactive group such as an isocyanate functional group. Non-limiting examples of suitable polyols include aliphatic, cycloaliphatic, aromatic, heterocyclic, oligomeric polyols, polymeric polyols, and mixtures thereof. For transparencies exposed to sunlight, aliphatic or cycloaliphatic polyols may be used. The number of carbon atoms in the polyol may range from 4 to 18, from 4 to 12, from 4 to 10, from 4 to 8, or from 4 to 6 carbon atoms. A carbon atom in the polyol may be replaced with a heteroatom, such as N, S, or O.

Non-limiting examples of trifunctional, tetrafunctional, or higher functional polyols suitable for use as the polyol include branched chain alkane polyols such as glycerol or glycerin, tetramethylolmethane, trimethylolethane (e.g., 1,1,1-trimethylolethane), trimethylolpropane (TMP; e.g., 1,1,1-trimethylolpropane), erythritol, pentaerythritol, dipentaerythritol, tripentaerythritol, sorbitan, alkoxylated derivatives thereof, and mixtures thereof. The polyol may be a cycloalkane polyol, such as trimethylene bis(1,3,5-cyclohexanetriol), and/or the polyol may be an aromatic polyol, such as trimethylene bis(1,3,5-benzenetriol).

The blue light protection transparency used in the display screen assembly of the present invention may be a rigid sheet or a film. By “rigid” is meant that the transparency possesses sufficient structural integrity to support a mechanical load equal to its own weight without cracking or deforming.

The transparency may be electrodimmable. For example, the transparency may include smart glass, which may also be referred to as switchable glass, a smart window, or a switchable window. The electrodimmable transparency may include any suitable device available in the art such as, for example, a suspended particle device, liquid crystal device, and/or an electrochromic window. The visible light transmittance of the electrodimmable transparency may be changed by the application of an electric current to the electrodimmable transparency. For example, the electrodimmable transparency may include a suspended particle device including a liquid suspension or film including opaque particles between two substrates coated with a transparent conductive material. When an electric current is applied to the transparent conductive material by a control device, the device is in a “light” state where the opaque particles have an ordered arrangement that allows light to pass through the device. In the absence of the electric current, the device is in a “dark” state where the opaque particles have a random arrangement and substantially block light from passing through the device. While the electrodimmable transparency may block light of a wavelength range of 400 nm to 430 nm while in the “dark” state, the electrodimmable transparency in the “dark” state also blocks visible light. The electrodimmable transparency may block greater than 50% of light of a wavelength range of 400 nm to 430 nm from passing through the electrodimmable transparency in both states and the electrodimmable transparency has a total visible light transmittance of greater than 65%, or a total visible light transmittance in the clear state that loses no more than 35% of the remaining light transmittance (e.g., if the electrodimmable transparency in the clear state has a total visible light transmittance of 50% without the additive, the total visible light transmittance of the electrodimmable transparency including the additive that blocks light having a wavelength of 400 nm to 430 nm would be at least 32.5%, based on a 35% reduction in the total visible light transmittance, which otherwise would have been 50%). The electrodimmable transparency may block greater than 70% of the light of the wavelength range of 400 nm to 430 nm from passing through the electrodimmable transparency and the electrodimmable transparency has a total visible light transmittance of greater than 60%. The additive of the present disclosure may be included in any suitable layer of the electrodimmable transparency (e.g., the suspended particle device).

In the preceding detailed description, only certain examples of the subject matter of the present disclosure are shown and described, by way of illustration. As those skilled in the art would recognize, the subject matter of the present disclosure may be embodied in many different forms and should not be construed as being limited to the description set forth herein. Also, in the context of the present disclosure, when a first element is referred to as being “on” a second element, it may be directly on the second element or be indirectly on the second element with one or more intervening elements interposed therebetween. Like reference numerals designate like elements throughout the specification. In the drawings, thicknesses may be exaggerated for convenience.

The word “comprising” and forms of the word “comprising” as used in this description and in the claims does not limit the recited subject matter to exclude any variants or additions. Although various features of the present disclosure have been described using the terms “comprising” or “including”, features consisting essentially of or consisting of are also within the scope of this disclosure. For example, while features of this disclosure have been described in terms of an additive chosen from inorganic nanoparticles, organic nanoparticles, organometallic nanoparticles, benzotriazoles, triazines, benzophenones, hindered amine light stabilizers, benzoates, cyanoacrylates, tetraphenylporphyrins, tetramesitylporphyrins, perylenes, phthalocyanines, chlorophylls, bilirubin, primary antioxidants, pigments, dyes, and combinations thereof, the additive consisting essentially of or consisting of any one of the inorganic nanoparticles, organic nanoparticles, organometallic nanoparticles, benzotriazoles, triazines, benzophenones, hindered amine light stabilizers, benzoates, cyanoacrylates, tetraphenylporphyrins, tetramesitylporphyrins, perylenes, phthalocyanines, chlorophylls, bilirubin, primary antioxidants, pigments, and/or dyes is also within the scope of this disclosure. In this context, “consisting essentially of” means that any additional components of the additive will not materially affect the light blockage properties (e.g., blockage of the light of the wavelength of 400 nm to 430 nm) of a transparency including the additive. As used herein, the term “plurality”, means two or more.

As used herein, unless otherwise expressly specified, all numbers such as those expressing values, ranges, amounts or percentages may be read as if prefaced by the word “about”, even if the term does not expressly appear. Plural encompasses singular and vice versa. For example, while features of the present disclosure have been described in terms of “an” additive, one or more of this or other recited components may be used according to the present disclosure. Also, as used herein, the term “polymer” is meant to refer to prepolymers, oligomers, and both homopolymers and copolymers; the prefix “poly” refers to two or more. When ranges are given, any endpoints of those and/or numbers within those ranges may be combined within the scope of the present disclosure. Including and like terms means “including but not limited to”. Similarly, as used herein, the terms “on” and “formed on” mean formed, overlaid, deposited, or provided on but not necessarily in contact with the surface, unless otherwise indicated. For example, an interlayer “formed on” a substrate does not preclude the presence of one or more other coating layers of the same or different composition located between the formed interlayer and the substrate, unless otherwise indicated. On the other hand, as used herein, the terms “directly on”, “formed directly on”, and “laminated directly on” mean in physical contact with the surface. For example, an interlayer directly on, formed directly on, or laminated directly on a substrate is in direct physical contact with the substrate. In addition, it will also be understood that when an element or layer is referred to as being “between” two elements or layers, it may be the only element or layer between the two elements or layers, or one or more intervening elements or layers may also be present.

Notwithstanding that the numerical ranges and parameters setting forth the broad scope of the present disclosure are approximations, numerical values set forth in the specific examples are reported as precisely as is practical. Any numerical value, however, may inherently contain certain errors necessarily resulting from the standard variation found in their respective testing measurements.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present disclosure.

Spatially relative terms, such as “beneath,” “below,” “lower,” “under,” “above,” “upper,” and the like, may be used herein for ease of explanation to describe one element or feature's relationship to another element(s) or feature(s) as illustrated in the figures. It will be understood that the spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” may encompass both an orientation of above and below. The device may be otherwise oriented (e.g., rotated 90 degrees or at other orientations) and the spatially relative descriptors used herein should be interpreted accordingly.

As used herein, the term “and/or” includes any and all combinations of one or more of the associated listed items. For example, the list “A, B, and/or C” is meant to encompass seven separate embodiments that include A, or B, or C, or A+B, or A+C, or B+C, or A+B+C. As used herein, the terms “substantially,” “about,” and similar terms are used as terms of approximation and not as terms of degree, and are intended to account for the inherent deviations in measured or calculated values that would be recognized by those of ordinary skill in the art. As used herein, the terms “use,” “using,” and “used” may be considered synonymous with the terms “utilize,” “utilizing,” and “utilized,” respectively.

Whereas particular embodiments of the present disclosure have been described herein for purposes of illustration, it will be evident to those skilled in the art that numerous variations of the details of the present disclosure may be made without departing from the invention as defined in the appended claims.

1. An electronic display screen assembly comprising a blue light protection transparency, said blue light protection transparency comprising an additive that blocks, reflects, refracts, and/or absorbs light having a wavelength of 380 nm to 430 nm, wherein the blue light protection transparency blocks greater than 50% of light of a wavelength range of 380 nm to 418 nm from passing through the blue light protection transparency as measured by ultraviolet-visible spectroscopy; and wherein the blue light protection transparency blocks 20% to 45% of light of a wavelength range of greater than 418 nm to 430 nm from passing through the blue light protection transparency as measured by ultraviolet-visible spectroscopy; and wherein the blue light protection transparency has a total visible light transmittance greater than 65% as measured using Procedure B according to ASTM D1003-07.

2. The electronic display screen assembly of claim 1, wherein the additive comprises at least one of a metal, a metal oxide, a benzotriazole, a triazine, a benzophenone, a hindered amine light stabilizer (HALS), a benzoate, a cyanoacrylate, a tetraphenylporphyrin, a tetramesitylporphyrin, a perylene, a oxalanilide, a phthalocyanine, chlorophyll including derivatives thereof, bilirubin including derivatives thereof, an antioxidant, a pigment, and a dye.

3. The electronic display screen assembly of claim 2, wherein the additive comprises a pyrrolo[3,4-f]benzotriazole-5,7(2H,6H)-dione.

4. The electronic display screen assembly of claim 3, wherein the pyrrolo[3,4-f]benzotriazole-5,7(2H,6H)-dione comprises 6-butyl-2-[2-hydroxy-3-(1-methyl-1-phenylethyl)-5-(1,1,3,3-tetramethylbutyl)phenyl] pyrrolo[3,4-f]benzotriazole-5,7(2H,6H)-dione, particularly in an amount of 0.01 wt % to 2.0 wt %, based on the total weight of the blue light protection transparency.

5. The electronic display screen assembly of claim 1, wherein the additive is combined with a resin and applied to a surface of the transparency in a light path of the electronic display screen assembly to form a coating layer thereon.

6. The electronic display screen assembly of claim 1, wherein the additive is incorporated into the blue light protection transparency.

7. The electronic display screen assembly of claim 6, wherein the additive includes a reactive functional group that facilitates incorporation of the additive into the transparency.

8. The electronic display screen assembly of claim 7, wherein the additive comprises Cu(II) Meso—tetra (4-carboxyphenyl) porphine.

9. The electronic display screen assembly of claim 1, wherein the blue light protection transparency comprises a polyurethane sheet.

10. The electronic display screen assembly of claim 9, wherein the polyurethane sheet comprises a reaction product of a reaction mixture comprising an isocyanate and an aliphatic polyol having 4 to 18 carbon atoms and at least 2 hydroxyl groups, wherein the isocyanate and the aliphatic polyol are independently branched or unbranched.

11. The electronic display screen assembly of claim 1, wherein the blue light protection transparency comprises glass.

12. The electronic display screen assembly of claim 1, wherein the display screen assembly comprises:
a first substrate;
a second substrate; and
an interlayer between the first substrate and the second substrate,
wherein the interlayer comprises the blue light protection transparency.

13. The electronic display screen assembly of claim 1, wherein the blue light protection transparency is situated as an outermost layer of the display screen assembly.

14. The electronic display screen assembly of claim 13, wherein the blue light protection transparency demonstrates a #0000 steel wool abrasion resistance of at least 2000 cycles using a 1 kg load, with a water contact angle greater than 100°.

15. The electronic display screen assembly of claim 1, wherein the blue light protection transparency is situated as an inner layer of the display screen assembly.

16. The electronic display screen assembly of claim 1, wherein the display screen assembly comprises an active or passive liquid crystal cell element; a light emitting diode; or a cathode ray tube.

17. The electronic display screen assembly of claim 16, wherein the display screen assembly comprises a display screen or a touch screen on a cell phone; tablet; GPS;
voting machine; interactive toy; digital camera; diagnostic tool; medical device; smart watch; gauge cluster; digital instrumentation display; POS (Point-Of-Sale) machine; a computer screen;
interactive advertising display screen; transparent see-through OLED display; electronic video display wall; smart appliance display screen; vehicle infotainment display screen; or a monitor screen.

18. The electronic display screen assembly of claim 17 wherein the display screen assembly comprises a display screen or a touch screen on a cell phone, tablet, GPS, or smart watch; a computer screen; or a monitor screen.

19. The electronic display screen assembly of claim 1, wherein the display screen assembly comprises an active or passive liquid crystal cell element and the blue light protection transparency comprises a polarizing layer.

20. An electronic device comprising the display screen assembly of claim 1.
